(19) United States
(12) Patent Application Publication
(10) Pub. No.: US 2010/0143543 A1
(43) Pub. Date: Jun. 10, 2010

(54) NUTRITIONAL SUPPLEMENT FORMULATIONS FOR INCLUSION IN FOODS AND FORTIFIED FOODS COMPRISING SUCH SUPPLEMENTS

(76) Inventors: Hans Hitz, Arisdorf (CH); Rolf Schaefer, Arisdorf (CH); Christoph Schaefer, Bubendorf (CH)

Correspondence Address: Richard Voellmy, Av de Sully 67, La Tour-de-Peitz, 1814, CH

(21) Appl. No.: 12/733,127

(22) PCT Filed: Sep. 15, 2008
(86) PCT No.: PCT/IB2008/002460

§ 371 Date: Feb. 12, 2010

Related U.S. Application Data
(60) Provisional application No. 60/960,171, filed on Sep. 19, 2007.

(51) Int. Cl. A23L 1/303; A23L 1/302

(52) U.S. Cl. 426/73; 426/72

(57) ABSTRACT

The present invention relates to formulations adapted from the original AREDS formula that are suitable for introduction into a measured amount or volume of a food as well as to foods containing these formulations. These fortified foods are expected to provide patients afflicted with or at risk for developing macular degeneration or other age-related deficiencies with alternative and more palatable sources of the ingredients of the original AREDS formula than those currently available in the market.

### FIELD OF THE INVENTION

The present invention relates to novel nutraceutical formulations comprising zinc, copper and other relevant metal ions, and anti-oxidants and to fortified foods containing such formulations in amounts believed to be effective in reducing the severity or slowing development of macular degeneration and preventing other age-related deficiencies.

### BACKGROUND OF THE INVENTION

The retina is the layer of nerve cells at the back of the eye, which nerve cells convert light into signals that are sent to the brain. In humans and other primates (but not in most other mammals), the macula is a small yellowish circular area in the retina, positioned at the center of the field of vision. It provides fine-resolution vision in the center of the visual field, and it is essential to good vision. Patients who suffer from macular degeneration often lose the ability to read, recognize faces, drive, or walk safely on unfamiliar routes. Macular degeneration is the leading cause of severe vision loss and functional blindness among the elderly, and its rates are increasing as the population ages and dietary patterns shift away from dark green vegetables toward more fatty foods. The disease, including approaches for preventing or treating it, is described in many books and articles. D'Amato R et al, Macular Degeneration: The Latest Scientific Discoveries and Treatments for Preserving Your Sight (Walker & Company, 2000); Lim J I, editor, Age-Related Macular Degeneration (Marcel Dekker, 2002); Byrne S et al, “Current concepts and recent advances in the management of age-related macular degeneration,” Ir J Med Sci 2003 October-December; 172(4): 185-90; Blodi B A, “Nutritional supplements in the prevention of age-related macular degeneration,” Insight 2004 January-March; 29(1): 15-6; Zarbin M A, “Current concepts in the pathogenesis of age-related macular degeneration,” Arch Opthalmol 2004 April; 122(4): 598-614.

While medications for treating macular degeneration have been developed (e.g., Lucentis™, approved in 2006 by the US FDA for vascular age-related macular degeneration) and continue to be developed, interest has remained high in the use of nutritional supplements for preventing or reducing progression of the disease. This interest dates back to the 1990s when the US National Eye Institute organized and carried out a major clinical trial involving thousands of patients who were suffering from (or were at high risk of developing) macular degeneration and/or cataracts. Macular degeneration and cataracts are clearly related to aging. They strongly increase in frequency and severity as people pass beyond the age of about 60, and they rarely if ever occur in anyone under the age of about 50, except in people who have specific genetic defects affecting their eyes. Accordingly, the trial was called the “Age-Related Eye Disease Study”, abbreviated as AREDS. As a follow-up study that likely will be called AREDS-2 is being discussed, the study carried out in the 1990's is referred to herein as AREDS-1. In the AREDS-1 study, participants were divided into four treatment arms, which were anti-oxidants alone, zinc alone, anti-oxidants plus zinc, or nothing (controls). In each treatment arm, participants were divided into four categories, depending on their eye health when they entered the study. Category 1, at the low end of the scale, contained people with no apparent signs of macular problems. Category 4, at the high end of the scale, contained people with serious problems in one eye while the other eye remained sufficiently free of advanced problems which permitted monitoring and measuring of subsequent declines after the person began taking supplements. Daily anti-oxidants administered were 500 mg of vitamin C, 265 mg of vitamin E and 15 mg of beta-carotene. Daily “zinc” was given as 80 mg of zinc in the form of zinc oxide in combination with 2 mg of copper in the form of cupric oxide for preventing anemia.

The main results of AREDS-1 were published in two articles in the October 2001 issue of Archives of Opthalmology. Macular degeneration data were reported in AREDS Report Number 8 (Arch Opthalmol 119: 1417-1436), whereas cataract data were reported in AREDS Report Number 9 (Arch Opthalmol 119: 1439-1452). While AREDS Report 8 should be consulted for details, its results generally can be summarized as follows: (1) Test patients who suffered from moderate or advanced macular degeneration, and who received Vitamins A, C, and E but no zinc, showed some positive results, but the indicators did not rise to a level of statistical significance. (2) Test patients who suffered from moderate or advanced macular degeneration, and who received zinc but no antioxidant vitamins, also showed some positive results, but the indicators did not reach statistical significance. (3) A third set of test patients who suffered from moderate or advanced macular degeneration, and who received both zinc and antioxidants, showed positive results that reached a level of statistical significance. U.S. Pat. No. 6,660,297 by Bartels et al., which patent is assigned to Bausch & Lomb, claims certain nutritional supplements that are based on the compositions tested in AREDS-1. The product being sold by Bausch & Lomb under that patent is called OCUVITE PRESERVISION™. A closely similar formulation called ICAPS AREDS™ is sold by Alcon Laboratories.

The safety of very high dosages of zinc and of vitamins C and E has been called into question. In addition to providing excessive levels of zinc and antioxidants that may be potentially dangerous, the current formulations are largely offered in the form of pills or gel caps, i.e., in formats typical for medications. Hence, these nutritional supplement formulations suffer from the same problem as typical medications, i.e., unsatisfactory patient compliance. To alleviate this problem, the present inventors have formulated relevant supplement mixtures as foods, including chocolate, the arguably most craved for food. In the course of this effort, problems needed to be resolved that related to oxidative instability of the supplement mixtures in non-solid state, and to inherent unpleasant taste and inhomogeneous distribution of supplement mixtures in foods. The present invention relates to new supplement mixtures as well as new foods containing such supplement mixtures.

### SUMMARY OF THE INVENTION

The present invention relates to formulations adapted from the original AREDS formula that are suitable for introduction into a measured amount or volume of a food. Measured amounts or volumes are also referred to as units and relate to amounts or volumes of fortified foods that are to be consumed daily by patients afflicted with macular degeneration or subjects at risk for developing the condition in order to obtain the expected health benefits of the formulations. The formulations for daily consumption comprise 90-2000 mg of vitamin C in the form of a fatty acid or ethyl ester, 15-1000 mg vitamin E, 11-40 mg zinc in the form of a fatty acid salt, 0.9-10 mg copper in the form of a fatty acid salt, and 1-25 mg lutein. Amounts of vitamin C indicated relate to unesterified ascorbic acid. Similarly, amounts given for zinc and copper (and other metals) are meant to be amounts of elemental zinc and copper (and metals). More preferred formulations comprise 200-600 mg of vitamin C in the form of a fatty acid or ethyl ester, 150-400 mg vitamin E, 11-40 mg zinc in the form of a fatty acid salt, 0.9-2.0 mg copper in the form of a fatty acid salt, and 1-15 mg lutein. In these formulations vitamin C can be added in the form of an ethyl, palmityl, lauryl, cocoyl, oleyl or stearyl ester. Zinc and copper each can be added either as palmitate, laureate, cocoate, oleate or stearate salts. Preferred are palmitate esters of vitamin C, and palmitate salts of zinc and copper. The most preferred formulation comprises 500 mg of vitamin C as palmityl ester, 265 mg of vitamin E, 25 mg zinc in the form of a palmitate salt, 1 mg of copper in the form of a palmitate salt and 2 mg lutein. Any of the aforementioned formulations may be supplemented with one or more additional compounds selected from the group consisting of vitamin A palmitate (20-40 mg) or beta-carotene (10-30 mg), omega-3-fatty acid triglyceride (50-200 mg), L-carnitin (25-100 mg), selenium palmitate (50-250 mcg) and zeaxanthin (5-25 mg). A related formulation supplemented with additional elements and vitamins comprises 500 mg vitamin C palmitate, 20 mg vitamin A palmitate, 265 mg vitamin D, 60 mg vitamin E, 25 mcg vitamin K, 6 mg vitamin 66, 25 mcg vitamin B12, 4.5 mg thiamine, 5 mg riboflavin, 40 mg niacin, 0.5 mg folic acid, 50 mcg biotin, 10 mg pantothenic acid, 0.15 mg iodine, 100 mg lecithin, 0.15 mg boron, 2 mg calcium stearate, 100 mg magnesium stearate, 25 mg zinc stearate, 2 mg copper stearate, 0.1 mg selenium stearate, 5 mg manganese stearate, 0.3 mg chromium stearate, 0.1 mg molybdenum stearate, 10 mcg nickel stearate, 20 mcg vanadium stearate, 10 mg silicium stearate, 0.5 mg lutein, 0.5 mg zeaxantin, 0.2 mg lycopene, 50 mg L-carnitin and 25 mg taurine. As for the formulation discussed previously, amounts given relate to elemental compounds and unesterified vitamins, respectively.

In another embodiment, the invention relates to a unit of a fortified food intended for daily consumption comprising 90-2000 mg of added vitamin C in the form of a fatty acid or ethyl ester, 15-1000 mg added vitamin E, 11-40 mg added zinc in the form of a fatty acid salt, 0.9-10 mg added copper in the form of a fatty acid salt, and 1-25 mg added lutein. Suitable liquid foods are milk and yoghurt, and suitable semisolid foods are chocolate, butter and margarine. Other foods that may be utilized as a base for preparing fortified foods are cream, ice cream, cereal bars and the like. More preferred is a fortified food for daily consumption comprising, per unit, 200-600 mg of added vitamin C in the form of a fatty acid or ethyl ester, 150-400 mg added vitamin E, 11-40 mg added zinc in the form of a fatty acid salt, 0.9-2.0 mg added copper in the form of a fatty acid salt, and 1-15 mg added lutein. Vitamin C can be supplemented in the form of an ethyl, palmityl, cocoyl, lauryl, oleyl or stearyl ester. Zinc and copper each can be added either as palmitate, cocoate, laureate, oleate or stearate salts. Preferred is the palmitate ester of vitamin C, and palmitate salts of zinc and copper. Most preferred is a fortified food for daily consumption supplemented, per unit, with 500 mg of vitamin C as palmityl ester, 265 mg of vitamin E, 25 mg zinc in the form of a palmitate salt, 1 mg of copper in the form of a palmitate salt and 2 mg lutein. Any of the aforementioned foods may be further supplemented with one or more additional compounds selected from the group consisting of vitamin A palmitate (20-40 mg) or beta-carotene (10-30 mg), omega-3-fatty acid triglyceride (50-200 mg), L-carnitin (25-100 mg), selenium palmitate (50-250 mcg) and zeaxanthin (5-25 mg). A related fortified food containing additional added elements and vitamins is supplemented, per unit, with 500 mg vitamin C palmitate, 20 mg vitamin A palmitate, 265 mg vitamin D, 60 mg vitamin E, 25 mcg vitamin K, 6 mg vitamin B6, 25 mcg vitamin B12, 4.5 mg thiamine, 5 mg riboflavin, 40 mg niacin, 0.5 mg folic acid, 50 mcg biotin, 10 mg pantothenic acid, 0.15 mg iodine, 100 mg lecithin, 0.15 mg boron, 2 mg calcium stearate, 100 mg magnesium stearate, 25 mg zinc stearate, 2 mg copper stearate, 0.1 mg selenium stearate, 5 mg manganese stearate, 0.3 mg chromium stearate, 0.1 mg molybdenum stearate, 10 mcg nickel stearate, 20 mcg vanadium stearate, 10 mg silicium stearate, 0.5 mg lutein, 0.5 mg zeaxantin, 0.2 mg lycopene, 50 mg L-carnitin and 25 mg taurine. As for the formulation discussed previously, amounts given relate to elemental compounds and unesterified vitamins, respectively.

A preferred specific embodiment relates to a single chocolate unit intended for daily consumption or a plurality of chocolate units, each unit comprising 90-2000 mg of added vitamin C in the form of a fatty acid or ethyl ester, 15-1000 mg added vitamin E, 11-40 mg added zinc in the form of a fatty acid salt, 0.9-10 mg added copper in the form of a fatty acid salt, and 1-25 mg added lutein. More preferred are a single chocolate unit or plurality of chocolate units, each unit comprising 200-600 mg of added vitamin C in the form of a fatty acid or ethyl ester, 150-400 mg added vitamin E, 11-40 mg added zinc in the form of a fatty acid salt, 0.9-2.0 mg added copper in the form of a fatty acid salt, and 1-15 mg added lutein. Vitamin C can be supplemented in the form of an ethyl, palmityl, cocoyl, lauryl, oleyl or stearyl ester. Zinc and copper each can be added either as palmitate, cocoate, laureate, oleate or stearate salts. Preferred are palmitate esters of vitamin C, and palmitate salts of zinc and copper. Most preferred is a chocolate unit or plurality of chocolate units for daily consumption, each unit supplemented with 500 mg of vitamin C as palmityl ester, 265 mg of vitamin E, 25 mg zinc in the form of a palmitate salt, 1 mg of copper in the form of a palmitate salt and 2 mg lutein. Any of the aforementioned chocolate chocolate units may be further supplemented with one or more additional compounds selected from the group consisting of vitamin A palmitate (20-40 mg) or beta-carotene (10-30 mg), omega-3-fatty acid triglyceride (50-200 mg), L-carnitin (25-100 mg), selenium palmitate (50-250 mcg) and zeaxanthin (5-25 mg). A related chocolate unit intended for daily consumption containing additional added elements and vitamins is supplemented with 500 mg vitamin C palmitate, 20 mg vitamin A palmitate, 265 mg vitamin D, 60 mg vitamin E, 25 mcg vitamin K, 6 mg vitamin B6, 25 mcg vitamin B12, 4.5 mg thiamine, 5 mg riboflavin, 40 mg niacin, 0.5 mg folic acid, 50 mcg biotin, 10 mg pantothenic acid, 0.15 mg iodine, 100 mg lecithin, 0.15 mg boron, 2 mg calcium stearate, 100 mg magnesium stearate, 25 mg zinc stearate, 2 mg copper stearate, 0.1 mg selenium stearate, 5 mg manganese stearate, 0.3 mg chromium stearate, 0.1 mg molybdenum stearate, 10 mcg nickel stearate, 20 mcg vanadium stearate, 10 mg silicium stearate, 0.5 mg lutein, 0.5 mg zeaxantin, 0.2 mg lycopene, 50 mg L-carnitin and 25 mg taurine. As for the formulation discussed previously, amounts given relate to elemental compounds and unesterified vitamins, respectively.

### DETAILED DESCRIPTION OF THE INVENTION

The present invention relates to new dosage forms of mixtures of zinc and antioxidants believed to be effective in retardation of progression or prevention of macular degeneration and other age-related deficiencies in elderly people. This belief is based on the results of the AREDS-1 study discussed before. Nutritional supplement formulations on the market are pills or gel caps, which are formulations that suffer from the same problem of unsatisfactory patient compliance as do many prescription and non-prescription drugs. It is presumed that regular consumption is a precondition for maximizing the medical benefits of the nutritional supplement. The inventors argue that patients will be more likely to comply if the nutritional supplement is provided in a food that is both highly attractive or even craved for and is devoid of any specific and unpleasant taste associated with components of the mixture, e.g., the acidic taste vitamin C and the burning and bitter tastes of heavy metal ions, and of specific texture due to uneven distribution of components, e.g., texture originating from pockets of insoluble metal oxides, etc.

As was mentioned before, the nutritional mixture that was administered daily in the AREDS-1 study was comprised of 500 mg of vitamin C, 265 mg of vitamin E, 15 mg of beta-carotene, 80 mg of zinc in the form of zinc oxide and 2 mg of copper in the form of cupric oxide. AREDS Report Number 8. Zinc and anti-oxidants are believed to be the active elements of the formula that protect against progression of macular degeneration. Copper is an essential trace metal. Copper deficiency results in anemia, cardiac abnormalities such as blood vessel and heart rupture, abnormal EKG's, and elevated levels of serum cholesterol, triglycerides and glucose. A lifetime of marginal dietary copper in humans is thought to lead to heart disease. Overt symptoms in adults are rare, but may occur with long-term shortage or, in those who regularly consume zinc supplements. Zinc is known to reduce copper status. Vitamin C supplementation also results in decreased copper status. In rats, large doses of vitamin C can lead to copper deficiency. Therefore, copper is included in the AREDS formula that comprises elevated concentrations of both zinc and vitamin C for preventing copper deficiency.

Presently marketed supplements contain the components of the AREDS formula in solid form. As soon as the chemicals are introduced into foods, which are compositions comprising water, interactions between the chemicals will occur. In particular, vitamin C is an anti-oxidant that will reduce copper ions to elemental, metallic copper, which is an unpleasant additive to foods, particularly when present in concentrated form. Moreover, metallic copper is in a different ionic state than the copper ions required by the AREDS formula. Perhaps most important, metallic copper is not bio-available. For intestinal absorption to occur, copper ions need to be bound by carrier proteins that are embedded in the intestinal lining. These carrier proteins, or ligands, hand off the copper ions to other larger carrier proteins located deeper within the intestinal wall. After several other steps, if all conditions are favorable, the ions are finally deposited on the other side of the intestinal wall, i.e., in the bloodstream, where they are available to the cells of the organism. Iron ions and iodine will also be reduced by vitamin C in aqueous environments.

Furthermore, when the original compounds present in the AREDS formula are introduced into a food, the food acquires a disagreeable taste due to the presence of vitamin C. Zinc and copper oxides are heavy and essentially water-insoluble and, consequently, are difficult to distribute homogeneously in a food.

The present inventors surprisingly found that fatty acid salts of copper were not reduced by vitamin C in an aqueous environment. Furthermore, copper ions were not reduced by fatty acid esters or ethyl esters of vitamin C. These observations provided the basis for creating liquid (e.g., milk, yoghurt) and semisolid foods (e.g., chocolate, cream, butter, margarine, ice cream, cereal bars, and the like) comprising essential components of the AREDS formula in their intended oxidation state.

Use of vitamin C in the form of a fatty acid ester eliminated the problem of unpleasant taste imparted on foods by vitamin C. Fatty acid salts of copper and zinc were found to be taste-neutral. In contrast to copper and zinc oxides, fatty acid salts of copper and zinc are readily dispersed in liquid and semi-solid foods. They distribute particularly well in highly lipophilic foods such as chocolates.

Very high dosages of vitamins C and E may not be safe. For example, studies have indicated that post-menopausal women who suffer from coronary artery disease should not take high dosages of vitamins C or E. In recent years, it has been realized that beta-carotene is not deposited in the macula in human eyes. Therefore, a consensus has emerged among eye and vision researchers that high dosages of beta-carotene do not offer any realistic promise of providing any substantial benefit or protection against macular degeneration. Moreover, beta-carotene actually reverses its anti-oxidant activity, and becomes a pro-oxidant, if unusually high concentrations of oxygen are present (e.g., Burton, G. W. et al. “Beta-carotene: An unusual type of lipid antioxidant,” Science 224: 569-573 (1984)). Such oxygen concentrations are not present in most tissues and fluids in the body; however, since the lungs interact directly with oxygen in air that is breathed, beta-carotene may act as a damaging pro-oxidant, rather than a beneficial anti-oxidant, in lung tissues. As an apparent result of its damaging pro-oxidant activity at high oxygen concentrations, large clinical trials have convincingly shown that high-dosage beta-carotene, instead of being useful and protective, actually increases the risks of lung cancer among smokers. This clearly and unmistakably occurs among smokers, and it may also be happening to a lesser extent among non-smokers. Accordingly, the Bartels et al. '297 patent specifically stated that beta-carotene could be deleted from the anti-oxidant combination that was tested in the AREDS-1 trial, and it could be replaced by either one or both of two other carotenoids, lutein and zeaxanthin, which carotenoids are actually present in the retina. Zinc ions were administered as zinc oxide in AREDS-1. Dosages used in the trial are believed to be 80 mg/day of zinc oxide; however, the published AREDS reports referred to “zinc, 80 mg, as zinc oxide”. This raises the question as to whether the “80 mg” dosage referred to elemental zinc (molecular weight 65.4), or zinc oxide (molecular weight 81.4). Current “Recommended Daily Allowance” values (abbreviated RDA; published in 2001 by the U.S. Food and Nutrition Board (part of the Institute of Medicine) for elemental zinc are 8 milligrams per day for adult females and 11 milligrams per day for adult males. Therefore, a daily dosage of 69.6 mg of elemental zinc, from OCUVITE PRESERVISION pills alone, without considering other dietary sources, is actually 870% of the RDA for women, and 633% of the RDA for men. In addition to RDA numbers, the Institute of Medicine has recently adopted and issued “Tolerable Upper Intake Levels” for various nutrients. For zinc, “Tolerable Upper Intake Levels” were set in 2001 at 40 mg/day for both men and women. Therefore, the amount of zinc in OCUVITE PRESERVISION, by itself, appears to be nearly twice as high as the “Tolerable Upper Intake Levels” (UL) set by the Institute of Medicine, and the surplus becomes even higher if additional zinc intake in the diet is also taken into account. As discussed in U.S. Pat. Publ. No. 20060039954 by Gierhart et al, there are numerous publications suggesting that extra-heavy dosages of zinc may cause serious risks, not among all elderly consumers, but among sufficient numbers to create major concerns. Three specific concerns involve neurology. The first focuses on Alzheimer's disease and beta-amyloid plaques. The second concern relates to the severity of brain damage and permanent impairment following a stroke, cardiac arrest, or other crisis that assaults the brain. The third concern focuses on cognitive impairments seen in animals that were fed heavy dosages of zinc. Heavy zinc intake may also trigger or stimulate the growth of prostate cancer among middle-aged and elderly men. In addition, zinc has been discovered in high concentrations in unwanted deposits in human retinas called “drusen”. This observation suggests the possibility that heavy zinc intake may accelerate the formation and growth of those unwanted deposits, which can disrupt the retina and damage vision. Apparently, the above risks were not recognized or considered by the people who organized and conducted the AREDS-1 trial or by the companies that are now selling formulations with heavy dosages of zinc to elderly consumers for the prevention or treatment of macular degeneration. It is argued that future formulations to be offered to elderly customers for the prevention or treatment of macular degeneration should have levels of zinc, vitamin C and vitamin E that do not exceed the UL values, and should include lutein or zeaxanthin instead of beta-carotene. As is argued in U.S. Pat. Publ. No. 20060039954, applying principles of homeostasis and diminishing marginal utility, such reduction of dosages of Zn, and vitamins C and E should not diminish markedly the ocular benefits of the formulations, and substitution of lutein or zeaxanthin for beta-carotene should enhance their effectiveness.

The present invention relates to formulations for daily consumption by adults comprising
(a) 90-2000 mg vitamin C as palmityl ester,
(b) 15-1000 mg vitamin E,
(c) 11-40 mg zinc as palmitate salt,
(d) 0.9-10 mg copper as palmitate salt, and
(e) 1-25 mg lutein

These formulations may be introduced into a food as a mixture, as individual components or as two or more mixtures of components.

### Preferred Formulations Comprise

(a) 200-600 mg vitamin C as palmityl ester,
(b) 150-400 mg vitamin E,
(c) 11-40 mg zinc as palmitate salt,
(d) 0.9-2 mg copper as palmitate salt, and
(e) 1-15 mg lutein.

These formulations may, in addition, be supplemented with one or more of the following ingredients (daily doses):
(f) 20-40 mg vitamin A palmitate or 10-30 mg beta-carotene,
(g) 50-200 mg omega-3-fatty acid-triglycerides,
(h) 25-100 mg carnitin,
(i) 50-250 mcg selenium palmitate, and
(j) 5-25 mg zeaxanthin.

### Most Preferred Formulations Comprise

(a) 500 mg vitamin C as palmityl ester,
(b) 265 mg vitamin E,
(c) 25 mg zinc as palmitate salt,
(d) 1 mg copper as palmitate salt, and
(e) 2 mg lutein,
   and may be further supplemented by the addition of one or more of the following ingredients:
(f) 20-40 mg vitamin A palmitate or 10-30 mg beta-carotene,
(g) 50-200 mg omega-3-fatty acid-triglycerides,
(h) 25-100 mg carnitin,
(i) 50-250 mcg mg selenium palmitate, and
(j) 5-25 mg zeaxanthin.

In the above formulations, vitamin C palmitate can be replaced by ethyl, lauryl, cocoyl, oleyl or stearyl esters of vitamin C. Furthermore, laureate, cocoate, oleate or stearate salts can be utilized instead of palmitate salts of zinc, copper, selenium and other heavy metal ions.

A more complete supplement formulation of the invention comprises
(a) 500 mg vitamin C-palmitate,
(b) 20 mg vitamin A-palmitate,
(c) 265 mg vitamin D,
(d)60 mg vitamin E,
(e) 25 mcg vitamin K,
(f) 6 mg vitamin B6,
(g) 25 mcg vitamin B12,
(h) 4.5 mg thiamin,
(i) 5 mg riboflavin,
(j) 40 mg niacin,
(k) 500 mcg folic acid,
(l) 50 mcg biotin,
(m) 10 mg pantothenic acid,
(n) 150 mcg iodine,
(o) 100 mg lecithin,
(p) 150 mcg boron,
(q) 2 mg calcium stearate,
(r) 100 mg magnesium stearate,
(s) 25 mg zinc stearate,
(t) 2 mg copper stearate,
(u) 100 mcg selenium stearate,
(v) 5 mg manganese stearate,
(w)300 mcg chromium stearate,
(x) 100 mcg molybdenum stearate,
(y) 10 mcg nickel stearate,
(z) 20 mcg vanadium stearate,
(aa) 10 mg silicium stearate,
(bb) 500 mcg lutein,
(cc) 500 mcg zeaxanthin,
(dd) 0.2 mg lycopene,
(ee) 50 mg L-carnitin, and
(ff) 25 mg taurine.

Again, palmitate, ethyl, lauryl, cocoyl, oleyl or stearyl esters may be utilized interchangeably. The same applies to palmitate, laureate, cocoate, oleate or stearate salts.

The present invention also relates to foods fortified with, in a an amount or volume of food to be consumed within a day, which amount or volume is referred to as a unit,
(a) 90-2000 mg vitamin C as palmityl ester,
(b) 15-1000 mg vitamin E,
(c) 11-40 mg zinc as palmitate salt,
(d) 0.9-10 mg copper as palmitate salt, and
(e) 1-25 mg lutein,
   more preferably
(a) 200-600 mg vitamin C as palmityl ester,
(b) 150-400 mg vitamin E,
(c) 11-40 mg zinc as palmitate salt,
(d) 0.9-2 mg copper as palmitate salt, and
(e) 1-15 mg lutein,
   optionally supplemented with one or more of the following ingredients
(f) 20-40 mg vitamin A palmitate or 10-30 mg beta-carotene,
(g) 50-200 mg omega-3-fatty acid-triglycerides,
(h) 25-100 mg carnitin,
(i) 50-250 mcg selenium palmitate, and
(j) 5-25 mg zeaxanthin,
   and most preferably,
(a) 500 mg vitamin C as palmityl ester,
(b) 265 mg vitamin E,
(c) 25 mg zinc as palmitate salt,
(d) 1 mg copper as palmitate salt, and
(e) 2 mg lutein,
   optionally supplemented with one or more of
(f) 20-40 mg vitamin A palmitate or 10-30 mg beta-carotene,
(g) 50-200 mg omega-3-fatty acid-triglycerides,
(h) 25-100 mg carnitin,
(i) 50-250 mcg selenium palmitate, and
(j) 5-25 mg zeaxanthin.

Vitamin C palmitate can be replaced by ethyl, lauryl, cocoyl, oleyl or stearyl esters of vitamin C. Furthermore, laureate, cocoate, oleate or stearate salts can be utilized instead of palmitate salts of zinc, copper and selenium.

A food of the invention may also fortified with a more complete supplement formulation of the invention comprising
(a) 500 mg vitamin C-palmitate,
(b) 20 mg vitamin A-palmitate,
(c) 265 mg vitamin D,
(d) 60 mg vitamin E,
(e) 25 mcg vitamin K,
(f) 6 mg vitamin B6,
(g) 25 mcg vitamin B12,
(h) 4.5 mg thiamin,
(i) 5 mg riboflavin,
(j) 40 mg niacin,
(k) 500 mcg folic acid,
(l) 50 mcg biotin,
(m)10 mg pantothenic acid,
(n) 150 mcg iodine,
(o) 100 mg lecithin,
(p) 150 mcg boron,
(q) 2 mg calcium stearate,
(r) 100 mg magnesium stearate,
(s) 25 mg zinc stearate,
(t) 2 mg copper stearate,
(u) 100 mcg selenium stearate,
(v) 5 mg manganese stearate,
(w)300 mcg chromium stearate,
(x) 100 mcg molybdenum stearate,
(y) 10 mcg nickel stearate,
(z) 20 mcg vanadium stearate,
(aa) 10 mg silicium stearate,
(bb) 500 mcg lutein,
(cc) 500 mcg zeaxanthin,
(dd) 0.2 mg lycopene,
(ee) 50 mg L-carnitin, and
(ff) 25 mg taurine

A preferred food for introduction of the formulations of the present invention is chocolate. Chocolates are not only the most craved for food, but they also contain high concentrations of flavonoids and other polyphenols that function as anti-oxidants. These anti-oxidants may have protective effects against age-related neurodegenerative diseases.

Chocolate is a product of cacao beans and has been prepared in various forms and consumed for millennia. To prepare chocolate, cacao beans harvested from pods of tropical cacoa trees (Theobroma cacao) are fermented for several days. Subsequent to drying, the beans are roasted, graded and ground. From the resulting chocolate liquor, cacao butter is removed either by being pressed or by the Broma process, i.e., by hanging a bag of ground cacao beans in a warm room. The cocoa butter drips off and is collected. This technique is now a common method for the production of cocoa and chocolate in the United States. The residue left behind after removal of cacao butter is known as cocoa powder.

Chocolate liquor is blended with the butter in varying quantities to make different types of chocolate or couvertures. The basic blends of ingredients, in order of highest quantity of cocoa liquor first, are as follows: (Note that since U.S. chocolates have a lower percentage requirement of cocoa liquor for dark chocolate, some dark chocolate may have sugar as the top ingredient.)
(a) Plain dark chocolate: sugar, cocoa butter, cocoa liquor, and (sometimes) vanilla
(b) Milk chocolate: sugar, cocoa butter, cocoa liquor, milk or milk powder, and vanilla
(c) White chocolate: sugar, cocoa butter, milk or milk powder, and vanilla.

The finest plain dark chocolate couvertures contain at least 70% cocoa (solids+butter), whereas milk chocolate usually contains up to 50%. High-quality white chocolate couvertures contain only about 33% cocoa.

To improve texture, an emulsifying agent such as soybean lecithin is added. Some manufacturers are now using polyglycerol polyricinoleate, an artificial emulsifier derived from castor oil. Texture is also heavily influenced by processing, specifically conching (see below). The more expensive chocolates tend to be processed longer and thus have a smoother texture and “feel” on the tongue, regardless of whether emulsifying agents are added.

The penultimate process is called conching. A conche is a container filled with metal beads, which act as grinders. The refined and blended chocolate mass is kept liquid by frictional heat. The conching process produces cocoa and sugar particles smaller than the tongue can detect, hence the smooth feel in the mouth. The length of the conching process determines the final smoothness and quality of the chocolate. High-quality chocolate is conched for about 72 hours, lesser grades about four to six hours. After the process is complete, the chocolate mass is stored in tanks heated to approximately 45-50° C. (113-122° F.) until final processing. The final process is called tempering. Uncontrolled crystallization of cocoa butter typically results in crystals of varying size, some or all large enough to be clearly seen with the naked eye. This causes the surface of the chocolate to appear mottled and matte, and causes the chocolate to crumble rather than snap when broken. The uniform sheen and crisp bite of properly processed chocolate are the result of consistently small cocoa butter crystals produced by the tempering process.

The fats in cocoa butter can crystallize in six different forms (polymorphous crystallization). The primary purpose of tempering is to assure that only the best form is present. The six different crystal forms have different properties.

| | Melting | |
|---|---|---|
| Crystal | Temp. | Notes |
| I | 17° C. (63° F.) | Soft, crumbly, melts too easily. |
| II | 21° C. (70° F.) | Soft, crumbly, melts too easily. |
| III | 26° C. (78° F.) | Firm, poor snap, melts too easily. |
| IV | 28° C. (82° F.) | Firm, good snap, melts too easily. |
| V | 34° C. (94° F.) | Glossy, firm, best snap, melts near body |
| | | temperature (37° C.). |
| VI | 36° C. (97° F.) | Hard, takes weeks to form. |

Making good chocolate is about forming the most of the type V crystals. This provides the best appearance and mouth feel and creates the most stable crystals so the texture and appearance will not degrade over time. To accomplish this, the temperature is carefully manipulated during the crystallization. The chocolate is first heated to 45° C. (113° F.) to melt all six forms of crystals. Then the chocolate is cooled to about 27° C. (80° F.), which will allow crystal types IV and V to form (VI takes too long to form). At this temperature, the chocolate is agitated to create many small crystal “seeds” which will serve as nuclei to create small crystals in the chocolate. The chocolate is then heated to about 31° C. (88° F.) to eliminate any type IV crystals, leaving just the type V. After this point, any excessive heating of the chocolate will destroy the temper and this process will have to be repeated. Two classic ways of tempering chocolate are:

(1) Working the melted chocolate on a heat-absorbing surface, such as a stone slab, until thickening indicates the presence of sufficient crystal “seeds”; the chocolate is then gently warmed to working temperature.
(2) Stirring solid chocolate into melted chocolate to “inoculate” the liquid chocolate with crystals (this method uses the already formed crystal of the solid chocolate to “seed” the melted chocolate).

A formulation of the invention can be introduced into chocolate at various stages of chocolate manufacture. Alternatively, a final chocolate product can be carefully molten to introduce a formulation of the invention.

Therefore, the present invention also relates to chocolate fortified with, in an amount to be consumed within a day, which amount is referred to herein as a unit,
(f) 90-2000 mg vitamin C as palmityl ester,
(g) 15-1000 mg vitamin E,
(h) 11-40 mg zinc as palmitate salt,
(i) 0.9-10 mg copper as palmitate salt, and
(j) 1-25 mg lutein,
   more preferably
(k) 200-600 mg vitamin C as palmityl ester,
(l) 150-400 mg vitamin E,
(m)11-40 mg zinc as palmitate salt,
(n) 0.9-2 mg copper as palmitate salt, and
(o) 1-15 mg lutein,
   optionally supplemented with one or more of the following ingredients
(p) 20-40 mg vitamin A palmitate or 10-30 mg beta-carotene,
(q) 50-200 mg omega-3-fatty acid-triglycerides,
(r) 25-100 mg carnitin,
(s) 50-250 mcg selenium palmitate, and
(t) 5-25 mg zeaxanthin,
   and most preferably,
(k) 500 mg vitamin C as palmityl ester,
(l) 265 mg vitamin E,
(m)25 mg zinc as palmitate salt,
(n) 1 mg copper as palmitate salt, and
(o) 2 mg lutein,
   optionally supplemented with one or more of
(p) 20-40 mg vitamin A palmitate or 10-30 mg beta-carotene,
(q) 50-200 mg omega-3-fatty acid-triglycerides,
(r) 25-100 mg carnitin,
(s) 50-250 mcg selenium palmitate, and
(t) 5-25 mg zeaxanthin.

Vitamin C palmitate can be replaced by ethyl, lauryl, cocoyl, oleyl or stearyl esters of vitamin C. Furthermore, laureate, cocoate, oleate or stearate salts can be utilized instead of palmitate salts of zinc, copper and selenium.

A chocolate unit of the invention may also fortified with a more complete supplement formulation of the invention comprising
(a) 500 mg vitamin C-palmitate,
(b) 20 mg vitamin A-palmitate,
(c) 265 mg vitamin D,
(d) 60 mg vitamin E,
(e) 25 mcg vitamin K,
(f) 6 mg vitamin B6,
(g) 25 mcg vitamin B12,
(h) 4.5 mg thiamin,
(i) 5 mg riboflavin,
(j) 40 mg niacin,
(k) 500 mcg folic acid,
(l) 50 mcg biotin,
(m)10 mg pantothenic acid,
(n) 150 mcg iodine,
(o) 100 mg lecithin,
(p) 150 mcg boron,
(q) 2 mg calcium stearate,
(r) 100 mg magnesium stearate,
(s) 25 mg zinc stearate,
(t) 2 mg copper stearate,
(u) 100 mcg selenium stearate,
(v) 5 mg manganese stearate,
(w)300 mcg chromium stearate,
(x) 100 mcg molybdenum stearate,
(y) 10 mcg nickel stearate,
(z) 20 mcg vanadium stearate,
(aa) 10 mg silicium stearate,
(bb) 500 mcg lutein,
(cc) 500 mcg zeaxanthin,
(dd) 0.2 mg lycopene,
(ee) 50 mg L-carnitin. and
(ff) 25 mg taurine.

The supplement mixtures of the invention may be distributed homogeneously in a chocolate unit. Alternatively, a supplement mixture may be distributed between two or more parts of a chocolate unit that may be presented to a patient separately or may be joined by Droste or Cresta processes. For example, the most preferred mixture may be introduced into two half chocolate units. The first half unit may be supplemented with 265 mg vitamin E and 8 mg lutein, and the second with 500 mg vitamin C as palmitate, and 30 mg zinc and 1 mg copper, both as palmitate salts. The half units may be joined by means of the Droste process.

A unit of a semisolid food, e.g., chocolate, butter or margarine, for daily consumption typically weighs from 5 to 20 g. A unit of a liquid food, e.g., milk or yoghurt, typically comprises a volume of between 20 and 200 ml.

Food units, e.g., chocolate, butter or margarine units, can be packaged separately. Alternatively, packages may contain multiple units sufficient for weekly, bi-weekly, monthly, etc., consumption.

### EXAMPLES

### Examples 1-4

50 mg vitamin C, 100 mg vitamin C as palmitate, 50 mg copper chloride or 100 mg copper as palmitate were dissolved separately at 50° C. in 1 ml water, milk, yoghurt or white chocolate. Formulations were then combined and incubated for 2 hours at appropriate temperatures. Reduction of copper II ions to elementary copper (metallic copper flakes) was monitored visually (Table 1).

TABLE 1
Reduction of copper ions in various food formulations

| | Vitamin C- & copper | Reduction to |
|---|---|---|
| Food | compounds | metallic copper |
| Water | Vitamin C & copper | Yes |
| | chloride | |
| | Vitamin C & copper as | No |
| | palmitate | |
| | Vitamin C as palmitate & | No |
| | copper chloride | |
| | Vitamin C as palmitate & | No |
| | copper as palmitate | |
| Milk | Vitamin C & copper | Yes |
| | chloride | |
| | Vitamin C & copper as | No |
| | palmitate | |
| | Vitamin C as palmitate & | No |
| | copper chloride | |
| | Vitamin C as palmitate & | No |
| | copper as palmitate | |
| Yoghourt | Vitamin C & copper | Yes |
| | chloride | |
| | Vitamin C & copper as | No |
| | palmitate | |
| | Vitamin C as palmitate & | No |
| | copper chloride | |
| | Vitamin C as palmitate & | No |
| | copper as palmitate | |
| White chocolate | Vitamin C & copper | Yes |
| | chloride | |
| | Vitamin C & copper as | No |
| | palmitate | |
| | Vitamin C as palmitate & | No |
| | copper chloride | |
| | Vitamin C as palmitate & | No |
| | copper as palmitate | |

### Examples 5-7

500 mg vitamin C, 265 mg vitamin E, 30 mg zinc as chloride salt, 1 mg copper as chloride salt and 2 mg lutein (mix A); 500 mg vitamin C as palmitate, 25 mg zinc as palmitate salt, 1 mg copper as palmitate salt and 2 mg lutein (mix B); or a more complete supplement formulation (mix C, see page 14/15) were added to 5 g of dark mint chocolate, white chocolate, margarine or butter, or 100 ml of strawberry yoghurt or milk. The resulting food formulations were tasted by a panel of 12 individuals. The tasting scale was defined as 1: expected food taste, 2: minor change of taste, 3: moderate change of taste, 4: significant change of taste, and 5: severe change of taste, not edible. Results are presented in Table 2 below.

TABLE 2
Taste testing of different fortified food formulations

| | Mint | | | | | |
|---|---|---|---|---|---|---|
| Supple- | choco- | White | | | Strawberry | |
| ment | late | chocolate | Margerine | Butter | yoghourt | Milk |
| Mix A | 4 | 5 | 5 | 5 | 5 | 5 |
| Mix B | 1 | 2 | 2 | 1 | 1 | 2 |
| Mix C | 1 | 2 | 1 | 2 | 1 | 2 |

1. A formulation for introduction into a unit of a food intended for daily consumption comprising 90-2000 mg of vitamin C in the form of a fatty acid or ethyl ester, 15-1000 mg vitamin E, 11-40 mg zinc in the form of a fatty acid salt, 0.9-10 mg copper in the form of a fatty acid salt, and 1-25 mg lutein.

2. The formulation of claim 1 wherein vitamin C is added in an amount of 200-600 mg in the form of a fatty acid or ethyl ester, vitamin E in an amount of 150-400 mg, zinc in an amount of 11-40 mg in the form of a fatty acid salt, copper in an amount of 0.9-2.0 mg in the form of a fatty acid salt, and lutein in an amount of 1-15 mg.

3. The formulation of claim 1 wherein vitamin C is added as an ethyl, palmityl, lauryl, cocoyl, oleyl or stearyl ester, and zinc and copper are added as palmitate, laureate, cocoate, oleate or stearate salts.

4. The formulation of claim 1 wherein vitamin C is added as a palmitate ester and zinc and copper as palmitate salts.

5. The formulation of claim 1 wherein vitamin C is present in an amount of 500 mg in the form of a palmityl ester, vitamin E in an amount of 265 mg, zinc in an amount of 25 mg in the form of a palmitate salt, copper in an amount of 1 mg in the form of a palmitate salt and lutein in an amount of 2 mg.

6. The formulation of claim 1 further comprising one or more additional ingredients selected from the group consisting of vitamin A palmitate (20-40 mg) or beta-carotene (10-30 mg), omega-3-fatty acid triglyceride (50-200 mg), L-carnitin (25-100 mg), selenium palmitate (50-250 mcg) and zeaxanthin (5-25 mg).

7. A formulation for introduction into a unit of a food intended for daily consumption, comprising 500 mg vitamin C palmitate, 20 mg vitamin A palmitate, 265 mg vitamin D, 60 mg vitamin E, 25 mcg vitamin K, 6 mg vitamin B6, 25 mcg vitamin B12, 4.5 mg thiamine, 5 mg riboflavin, 40 mg niacin, 0.5 mg folic acid, 50 mcg biotin, 10 mg pantothenic acid, 0.15 mg iodine, 100 mg lecithin, 0.15 mg boron, 2 mg calcium stearate, 100 mg magnesium stearate, 25 mg zinc stearate, 2 mg copper stearate, 0.1 mg selenium stearate, 5 mg manganese stearate, 0.3 mg chromium stearate, 0.1 mg molybdenum stearate, 10 mcg nickel stearate, 20 mcg vanadium stearate, 10 mg silicium stearate, 0.5 mg lutein, 0.5 mg zeaxantin, 0.2 mg lycopene, 50 mg L-carnitin and 25 mg taurine.

8. A unit of a fortified food intended for daily consumption comprising 90-2000 mg of added vitamin C in the form of a fatty acid or ethyl ester, 15-1000 mg added vitamin E, 11-40 mg added zinc in the form of a fatty acid salt, 0.9-10 mg added copper in the form of a fatty acid salt, and 1-25 mg added lutein.

9. The unit of a fortified food of claim 8 wherein vitamin C is added in an amount of 200-600 mg in the form of a fatty acid or ethyl ester, vitamin E in an amount of 150-400 mg, zinc in an amount of 11-40 mg in the form of a fatty acid salt, copper in an amount of 0.9-2.0 mg in the form of a fatty acid salt, and lutein in an amount of 1-15 mg.

10. The unit of a fortified food of claim 8 wherein vitamin C is added as an ethyl, palmityl, lauryl, cocoyl, oleyl or stearyl ester, and zinc and copper are added as palmitate, laureate, cocoate, oleate or stearate salts.

11. The unit of a fortified food of claim 8 wherein vitamin C is added as a palmitate ester, and zinc and copper as palmitate salts.

12. The unit of a fortified food of claim 8 wherein vitamin C is added in an amount of 500 mg in the form of a palmityl ester, vitamin E in an amount of 265 mg, zinc in an amount of 25 mg in the form of a palmitate salt, copper in an amount of 1 mg in the form of a palmitate salt and lutein in an amount of 2 mg.

13. The unit of a fortified food of claim 8 further supplemented with one or more additional ingredients selected from the group consisting of vitamin A palmitate (20-40 mg) or beta-carotene (10-30 mg), omega-3-fatty acid triglyceride (50-200 mg), L-carnitin (25-100 mg), selenium palmitate (50-250 mcg) and zeaxanthin (5-25 mg).

14. A unit of a fortified food intended for daily consumption supplemented with 500 mg vitamin C palmitate, 20 mg vitamin A palmitate, 265 mg vitamin D, 60 mg vitamin E, 25 mcg vitamin K, 6 mg vitamin B6, 25 mcg vitamin B12, 4.5 mg thiamine, 5 mg riboflavin, 40 mg niacin, 0.5 mg folic acid, 50 mcg biotin, 10 mg pantothenic acid, 0.15 mg iodine, 100 mg lecithin, 0.15 mg boron, 2 mg calcium stearate, 100 mg magnesium stearate, 25 mg zinc stearate, 2 mg copper stearate, 0.1 mg selenium stearate, 5 mg manganese stearate, 0.3 mg chromium stearate, 0.1 mg molybdenum stearate, 10 mcg nickel stearate, 20 mcg vanadium stearate, 10 mg silicium stearate, 0.5 mg lutein, 0.5 mg zeaxantin, 0.2 mg lycopene, 50 mg L-carnitin and 25 mg taurine.

15. The units of claim 8, wherein the fortified food is selected from the group consisting of butter, margarine, milk and yoghurt.

16. A single chocolate unit intended for daily consumption or a plurality of chocolate units, each unit comprising 90-2000 mg of added vitamin C in the form of a fatty acid or ethyl ester, 15-1000 mg added vitamin E, 11-40 mg added zinc in the form of a fatty acid salt, 0.9-10 mg added copper in the form of a fatty acid salt, and 1-25 mg added lutein.

17. The single chocolate unit or plurality of chocolate units of claim 16, wherein each unit comprises 200-600 mg of added vitamin C in the form of a fatty acid or ethyl ester, 150-400 mg added vitamin E, 11-40 mg added zinc in the form of a fatty acid salt, 0.9-2.0 mg added copper in the form of a fatty acid salt, and 1-15 mg added lutein.

18. The single chocolate unit or plurality of chocolate units of claim 16 wherein in each unit vitamin C is added as an ethyl, palmityl, lauryl, oleyl or stearyl ester, and zinc and copper are added as palmitate, laureate, oleate or stearate salts.

19. The single chocolate unit or plurality of chocolate units of claim 16 wherein in each unit vitamin C is added as a palmitate ester and zinc and copper as palmitate salts.

20. The single chocolate unit or plurality of chocolate units of claim 16 wherein in each unit vitamin C is added in an amount of 500 mg in the form of a palmityl ester, vitamin E in an amount of 265 mg, zinc in an amount of 25 mg in the form of a palmitate salt, copper in an amount of 1 mg in the form of a palmitate salt and lutein in an amount of 2 mg.

21. The single chocolate unit or plurality of chocolate units of claim 16 wherein each unit is further supplemented with one or more additional ingredients selected from the consisting of vitamin A palmitate (20-40 mg) or beta-carotene (10-30 mg), omega-3-fatty acid triglyceride (50-200 mg), L-carnitin (25-100 mg), selenium palmitate (50-250 mcg) and zeaxanthin (5-25 mg).

22. A single chocolate unit for daily consumption or a plurality of chocolate units, each unit supplemented with 500 mg vitamin C palmitate, 20 mg vitamin A palmitate, 265 mg vitamin D, 60 mg vitamin E, 25 mcg vitamin K, 6 mg vitamin B6, 25 mcg vitamin B12, 4.5 mg thiamine, 5 mg riboflavin, 40 mg niacin, 0.5 mg folic acid, 50 mcg biotin, 10 mg pantothenic acid, 0.15 mg iodine, 100 mg lecithin, 0.15 mg boron, 2 mg calcium stearate, 100 mg magnesium stearate, 25 mg zinc stearate, 2 mg copper stearate, 0.1 mg selenium stearate, 5 mg manganese stearate, 0.3 mg chromium stearate, 0.1 mg molybdenum stearate, 10 mcg nickel stearate, 20 mcg vanadium stearate, 10 mg silicium stearate, 0.5 mg lutein, 0.5 mg zeaxantin, 0.2 mg lycopene, 50 mg L-carnitin and 25 mg taurine.
